SEQUENCE LISTING (6 sequences)

SEQ ID NO: 1 - DNA, 20 bp, Artificial Sequence
  Forward primer
tgtgtccgtcgtggatctga

SEQ ID NO: 2 - DNA, 21 bp, Artificial Sequence
  Reverse primer
cctgcttcaccaccttcttga

SEQ ID NO: 3 - DNA, 23 bp, Artificial Sequence
  Forward primer
ctgcaagagacttccatccagtt

SEQ ID NO: 4 - DNA, 20 bp, Artificial Sequence
  Reverse primer
aagtagggaaggccgtggtt

SEQ ID NO: 5 - DNA, 21 bp, Artificial Sequence
  Forward primer
tgctatgctgcctgctcttac

SEQ ID NO: 6 - DNA, 22 bp, Artificial Sequence
  Reverse primer
cggttagcagtatgttgtccag

(19) United States
(12) Patent Application Publication
(10) Pub. No.: US 2020/0254033 A1
(43) Pub. Date: Aug. 13, 2020

(54) COMPOSITION FOR PREVENTING AND IMPROVING GASTROINTESTINAL DISORDER CONTAINING LACTOBACILLUS PLANTARUM

(71) Applicants: Amorepacific Corporation, Seoul (KR); HANDONG GLOBAL UNIVERSITY INDUSTRY-ACADEMIC COOPERATION FOUNDATION, Pohang-si, Gyeongsangbuk-do (KR)

(72) Inventors: Juyeon SEO, Yongin-si (KR); Donghyun CHO, Yongin-si, (KR); Wangi KIM, Yongin-si (KR); Wilhelm HOLZAPFEL, Pohang-si (KR); Yosep JI, Pohang-si (KR); Haryung PARK, Pohang-si (JP); Hyeonrae KIM, Pohang-si (KR)

(73) Assignees: Amorepacific Corporation, Seoul (KR); HANDONG GLOBAL UNIVERSITY INDUSTRY-ACADEMIC COOPERATION FOUNDATION, Pohang-si, Gyeongsangbuk-do (KR)

(21) Appl. No.: 16/647,250

(22) PCT Filed: Aug. 9, 2018
(86) PCT No.: PCT/KR2018/009089

§ 371 Date: Mar. 13, 2020

(30) Foreign Application Priority Data
Sep. 15, 2017 (KR) 10-2017-0118427

(51) Int. Cl. A61K 35/747; C12R 1/25; A61P 1/00; A23L 33/135; A23L 33/00; A23L 2/52

(52) U.S. Cl. CPC A61K 35/747; C12R 1/25; A61P 1/00; A23Y 2220/67; A23L 33/40; A23L 2/52; A23V 2002/00; A23L 33/135

(57) ABSTRACT

A composition including at least one selected from the group consisting of the APsulloc 331261 (accession number: KCCM11179P) strain of Lactobacillus plantarum, a lysed product thereof, a culture thereof, and an extract thereof is disclosed. The composition has an activity of improving or treating disorders of the digestive system caused by alcohol or inflammation to facilitate the recovery of and improve the welfare of relevant patients. A method of preventing, improving, or treating gastrointestinal disorders by using the composition is disclosed.

### TECHNICAL FIELD

The present specification relates to a composition for preventing, improving, or treating gastrointestinal disorders containing Lactobacillus plantarum.

### BACKGROUND ART

The stomach is an organ that stores food entering through the esophagus, breaks down the food into smaller pieces for easy digestion, and controls the sending of food to the duodenum in harmony with the secretion of various enzymes for efficient digestion and absorption. The stomach secretes gastric acid to digest food when food enters. In this case, a protective layer of gastric mucosa acts to prevent the gastric mucosa from being damaged by gastric acid. The protective layer of gastric mucosa that protects the stomach is susceptible to attacks and damage from various types of factors. Representative aggressive factors include gastric acid, alcohol, nonsteroidal anti-inflammatory drugs (NSAID) such as aspirin, bacteria such as Helicobacter pylori, microcirculation disorders of gastric mucosa induced by stress, and hypotension. When these factors damage the gastric mucosal layer, inflammation (gastritis) accompanied by erosion, redness, bleeding, and edema occurs, leading to gastric ulcer when the damage is severe to an extent that the lower mucosa and muscularis are also damaged by penetration of the gastric mucosa. In addition, when the duodenal mucosa is damaged by such factors, the duodenitis may proceed, and when the damage penetrates further than the most superficial mucosa and proceeds beyond the muscularis mucosa, the duodenal ulcer occurs.

At present, the most representative therapeutic agents for gastrointestinal disorders are antacids that neutralize excessive gastric secreting fluid, histamine H2 receptor antagonists and proton pump inhibitors that inhibit gastric acid secretion, prostaglandin that increases resistance of neointima against digestive fluids and inhibits acid secretion, or gastric mucosal protective agents, etc.

The main symptoms of gastrointestinal disorders such as gastritis, gastric ulcer, duodenitis, and duodenal ulcer include abdominal pain, heartburn, bloating, indigestion, burping, nausea, vomiting and bleeding. Antacids are taken to relieve these symptoms, but antacids merely have a fast-acting effect of temporarily neutralizing gastric acid, and thus have the disadvantages that it should be administered continuously and of side effects such as constipation, diarrhea, and urolithiasis. Drugs that block the secretion of gastric acid may be used. Acetylcholine receptor inhibitors and prostaglandin receptor active drugs are not widely used because they act on nerve cells other than the gastrointestinal tract and show side effects. Drugs having a hydrogen ion carrier inhibitory mechanism have also been reported to have a problem of thickening of the stomach wall when taken for a long time. In addition, steroidal anti-inflammatory drugs are limited in use due to various side effects such as immunodeficiency. Nonsteroidal anti-inflammatory drugs, especially aspirin and ibuprofen, etc., rather aggravate gastric or duodenal ulcers, and thus have been limited in their use in patients with ulcers such as being extremely contraindicated or taken with antacids. In addition, antibiotics or fungistats that inhibit Helicobacter pylori bacteria are known to have side effects such as diarrhea, abdominal pain and nausea, etc., and are inconvenient to ingest due to their unique bitter taste.

Accordingly, it is urgent to develop new products that alleviate the symptoms of gastrointestinal disorders and have fewer side effects even after long-term use.

### SUMMARY OF INVENTION

### Technical Problem

One aspect of the present disclosure is directed to developing a new product capable of improving and treating gastrointestinal disorders with fewer side effects even after long-term use.

### Solution to Problem

One aspect of the present disclosure provides a composition for preventing, improving, or treating gastrointestinal disorders including at least one selected from the group consisting of the APsulloc 331261 (accession number: KCCM11179P) strain of Lactobacillus plantarum, a lysed product thereof, a culture thereof, and an extract thereof.

### Advantageous Effects of Invention

In one aspect of the present disclosure, the composition can be provided to improve or treat disorders of the digestive system caused by alcohol or inflammation to facilitate the recovery of and improve the welfare of relevant patients, and contribute to the development of the food and drug industry relating to the disorders.

### BRIEF DESCRIPTION OF DRAWINGS

FIG. 1 is a result of photographing gastrointestinal tissue obtained by dividing mice into five groups and experimenting and dissecting these groups, and shows Group 1 treated with only PBS (phosphate buffer saline), Group 2 treated with PBS and ethanol, Group 3 treated with ethanol and L. plantarum APsulloc 331261, Group 4 treated with ethanol and L. plantarum 299V, and Group 5 treated with Omeprazole (OMPZ).

FIG. 2 is a graph confirming IL6/IL10 in the gastrointestinal tissue of the groups each treated with PBS, ethanol, L. plantarum APsulloc 331261, L. plantarum 299V, and omeprazole.

FIG. 3 shows a microscopic observation of the gastrointestinal tissue of each group in FIG. 2.

### DESCRIPTION OF EMBODIMENTS

The inventors of the present disclosure have attempted to develop a new composition for preventing and treating gastrointestinal disorders that has fewer side effects than conventional chemical drugs and secured stability. In order to prevent and treat gastrointestinal disorders such as gastritis, gastric ulcer, esophagitis, esophageal ulcer, duodenitis, and duodenal ulcer, etc. caused by various aggressive factors, it is necessary to protect the gastrointestinal tract from such aggressive factors and an action is needed such as anti-inflammation. To this end, the inventors concentrated on research, focusing on lactic acid bacteria.

Lactic acid bacteria refer to bacteria that produce lactic acid by breaking down sugars such as glucose and are also referred to as lactobacillus. This property of lactic acid bacteria is used for producing foods such as dairy products, kimchi, and brewed foods. In addition, lactic acid bacteria are also used as an intestinal drug as they live in the intestines of mammals to prevent abnormal fermentation caused by various bacteria. Lactobacillus plantarum is a strain belonging to lactic acid bacteria and is known to grow mainly when kimchi is fermented a lot and tastes sour. Lactobacillus plantarum is rod-shaped bacilli and gram-positive bacteria, and optical isomers of the produced lactic acid are D-type and L-type.

Tea for drinking, on the other hand, is produced by inactivating oxidase present in sprouts or leaves of theaceous Camellia sinensis and removing moisture. These teas contain caffeine, tannins, flavonoids, and essential oils as well as vitamins, and have been used in the food field.

The present inventors have found that the Lactobacillus plantarum APsulloc 331261 strain (accession number: KCCM11179P) isolated from the leaves of Jeju Camellia sinensis has an effect of improving gastrointestinal disorders, thus completed one aspect of the present disclosure.

As used in one aspect of the present disclosure, the term “gastrointestinal disorder” refers to disorders of organs, including the stomach and duodenum, and includes esophageal disorders such as esophagitis and esophageal ulcer.

In one aspect, the present disclosure may provide a composition for preventing, improving, or treating gastrointestinal disorders including at least one selected from the group consisting of the APsulloc 331261 (accession number: KCCM11179P) strain of Lactobacillus plantarum, a lysed product thereof, a culture thereof, and an extract thereof.

The APsulloc 331261 strain is a strain isolated from the leaves of Camellia sinensis and belongs to Lactobacillus plantarum. Specifically, the APsulloc 331261 strain may be isolated by a method including: salting the leaves of Camellia sinensis with table salt of 5 to 15% by weight relative to the weight of the leaves of Camellia sinensis; mixing the salted leaves of Camellia sinensis with a sugar solution, for example, 0.1% to 3% of fructo oligosaccharides, followed by culturing at 25° C. to 35° C. for 1 to 5 days; and collecting the culture solution at less than pH 5, followed by culturing at anaerobic condition of 25° C. to 35° C. for 1 to 5 days.

In one aspect of the present specification, the culture solution or extract thereof may refer to a culture solution containing a culture product generated by culturing a strain, for example, Lactobacillus plantarum APsulloc 331261, or an extract of the culture. The culture solution may also contain in the strain itself.

In one embodiment, the gastrointestinal disorder may be an esophageal, gastrointestinal or duodenal disorder caused by any one or more of alcohol, food containing alcohol, and alcoholic beverage.

In another embodiment, the alcohol may be ethanol.

In another embodiment, the gastrointestinal disorder may be at least one selected from the group consisting of gastritis, gastric ulcer, esophagitis, esophageal ulcer, duodenitis, and duodenal ulcer.

In another embodiment, the gastrointestinal disorder may be one in which the ratio of IL (interleukin receptor) 6/IL10 expression level in one or more tissues of the esophagus, stomach, and duodenum is higher than that of a normal person. The IL6/IL10 ratio is an indicator known to increase in gastrointestinal stress situations such as gastritis, duodenitis, esophagitis, gastrointestinal tract ulcer and gastric cancer.

When the APsulloc 331261 strain of Lactobacillus plantarum, a lysed product thereof, a culture thereof, or an extract thereof of one aspect of the present specification is administered, it was found that there is an effect that inflammation and bleeding are reduced in gastrointestinal tissues, and the IL6/IL10 ratio is significantly reduced (see Experimental Examples 1 and 2). Accordingly, according to one aspect, the present disclosure can be used for improvement and treatment for such disorders, and in particular, one aspect of the present disclosure can be applied to gastrointestinal disorders (gastritis, gastric ulcer, esophagitis, esophageal ulcer, duodenitis, and duodenal ulcer) caused by alcohol.

In one embodiment, the daily dosage of the composition may be 1×10³ CFU/day or more, 1×10⁴ CFU/day or more, 1×10⁵ CFU/day or more, 1×10⁶ CFU/day or more, 1×10⁷ CFU/day or more, 1×10⁸ CFU/day or more, 1×10⁹ CFU/day or more, 1×10¹⁰ CFU/day or more, 1×10¹¹ CFU/day or more, or 1×10¹² CFU/day or more. In another embodiment, the daily dosage may be 1×10¹³ CFU/day or less, 1×10¹² CFU/day or less, 1×10¹¹ CFU/day or less, 1×10¹⁰ CFU/day or less, 1×10⁹ CFU/day or less, 1×10⁸ CFU/day or less, 1×10⁷ CFU/day or less, 1×10⁶ CFU/day or less, 1×10⁵ CFU/day or less, or 1×10⁴ CFU/day or less. The daily dosage may be, for example, 10⁶ to 10¹⁰ CFU/day, but is not limited thereto, and may vary due to various factors such as age, health condition, complications, etc. of a subject to be administered.

The APsulloc 331261 strain of Lactobacillus plantarum, a lysed product thereof, a culture thereof, or an extract thereof according to one aspect of the present disclosure may be contained in an amount of 0.01% to 10% by weight based on the total weight of the composition. In one specific embodiment, the APsulloc 331261 strain of Lactobacillus plantarum, a lysed product thereof, a culture thereof, or an extract thereof according to one aspect of the present disclosure may be contained in an amount of 0.1% to 5% by weight based on the total weight of the composition. In the above aspect, the APsulloc 331261 strain of Lactobacillus plantarum, a lysed product thereof, a culture thereof, or an extract thereof according to one aspect of the present disclosure may be contained in an amount of 0.001% by weight or more, 0.005% by weight or more, 0.01% by weight or more, 0.1% by weight or more, 1% by weight or more, 3% by weight or more, 5% by weight or more, 7% by weight or more, 9% by weight or more, 10% by weight or more, or 11% by weight or more, and 11% by weight or less, 10% by weight or less, 9% by weight or less, 7% by weight or less, 5% by weight or less, 3% by weight or less, 1% by weight or less, 0.1% by weight or less, 0.005% by weight or less, or 0.003% by weight or less based on the total weight of the composition.

As another embodiment, the route of administration of the composition may be oral administration, but the composition may be directly administered to the abdominal cavity, or the esophagus and the gastrointestinal tract, or may be administered by various methods such as simple intake, drinking, injection administration, spray administration or squeeze administration.

In one embodiment, the composition may be a food composition, a health functional food composition, or a pharmaceutical composition.

In another aspect, the present disclosure may relate to a method for preventing, improving, or treating gastrointestinal disorders comprising administering at least one selected from the group consisting of the APsulloc 331261 (accession number: KCCM11179P) strain of Lactobacillus plantarum, a lysed product thereof, a culture thereof, and an extract thereof to a subject in need of prevention, improvement or treatment of gastrointestinal disorders. In an aspect of the present disclosure, the administration of the method may be carried out according to the administration method and administration amount described herein.

In another aspect, the present disclosure may relate to a use of a combination comprising at least one selected from the group consisting of the APsulloc 331261 (accession number: KCCM11179P) strain of Lactobacillus plantarum, a lysed product thereof, a culture thereof, and an extract thereof for the preparation of a food composition for the prevention, improvement or treatment of gastrointestinal disorders.

In another aspect, the present disclosure may relate to a use of a combination comprising at least one selected from the group consisting of the APsulloc 331261 (accession number: KCCM11179P) strain of Lactobacillus plantarum, a lysed product thereof, a culture thereof, and an extract thereof for the preparation of a health functional food composition for the prevention, improvement or treatment of gastrointestinal disorders.

In another aspect, the present disclosure may relate to a use of a combination comprising at least one selected from the group consisting of the APsulloc 331261 (accession number: KCCM11179P) strain of Lactobacillus plantarum, a lysed product thereof, a culture thereof, and an extract thereof for the preparation of a pharmaceutical composition for the prevention, improvement or treatment of gastrointestinal disorders.

In another aspect, the present disclosure may relate to a use for preventing, improving, or treating gastrointestinal disorders of a combination comprising at least one selected from the group consisting of the APsulloc 331261 (accession number: KCCM11179P) strain of Lactobacillus plantarum, a lysed product thereof, a culture thereof, and an extract thereof.

In another aspect, the present disclosure may relate to a use of a combination comprising at least one selected from the group consisting of the APsulloc 331261 (accession number: KCCM11179P) strain of Lactobacillus plantarum, a lysed product thereof, a culture thereof, and an extract thereof for the prevention, improvement, or treatment of gastrointestinal disorders.

As another embodiment, the formulation of the food or health functional food may be powder or liquid, but is not particularly limited thereto. For example, the food or health functional food may include tablets, granules, pills, powders, capsules, liquids such as drinks, and any form of processed food formulations, such as caramels, sol, gels, bars, and tea bags, and may be formulated into health functional food including vitamins and minerals, etc. The composition of each formulation may be mixed by appropriately selecting the ingredients commonly used in the pertinent field by those skilled in the pertinent field without any difficulty according to the formulation or purpose of use. Synergistic effects may occur when applied simultaneously with other raw materials.

In an aspect of the present disclosure, the food or health functional food composition may contain various nutrients, vitamins, minerals (electrolytes), flavoring agents including synthetic and natural flavoring agents, colorants, extenders (cheese and chocolate, etc.), pectic acid and salts thereof, alginic acid and salts thereof, organic acids, protective colloidal thickeners, pH control agents, stabilizers, preservatives, glycerin, alcohols, and carbonating agents used in soft drinks. In addition, in one aspect of the present disclosure, the food composition may contain a pulp for preparing a natural fruit juice, a fruit drink and a vegetable drink. These ingredients may be used either independently or in combination. The ratio of the additives is of no significant importance, but in one aspect of the present disclosure, the additives may generally be contained in an amount of about 0-50 parts by weight based on 100 parts by weight of the composition.

In one aspect of the present disclosure, the pharmaceutical composition may be a variety of oral or parenteral formulations. The pharmaceutical composition may be prepared into a formulation using a commonly used diluent or excipient such as a filler, an extender, a binder, a wetting agent, a disintegrating agent, and a surfactant, etc. Solid formulations for oral administration may include a tablet, a pill, a powder, a granule, and a soft or hard capsule, etc., and these formulations are prepared by mixing at least one excipient, such as starch, calcium carbonate, sucrose or lactose, and gelatin, etc. In addition to the simple excipient, a lubricant such as magnesium stearate and talc may also be used. Liquid formulations for oral administration may include a suspension, a liquid medicine for internal use, an emulsion, and a syrup, etc. In addition to a commonly used simple diluent such as water or liquid paraffin, various excipients such as a humectant, a sweetener, a flavoring agent, and a preservative, etc. may also be contained. Formulations for parenteral administration may include a sterilized aqueous solution, a nonaqueous solution, a suspension, an emulsion, a freeze-dried product and a suppository. The nonaqueous solution or suspension may contain propylene glycol, polyethylene glycol, vegetable oil such as olive oil, and an injectable ester such as ethyl oleate, etc. As a base of the suppository, witepsol, macrogol, tween 61, cocoa butter, laurin butter, and glycerogelatin, etc. may be used.

In one aspect of the present disclosure, the pharmaceutical composition may be administered by a method such as oral administration, or injection administration into the abdominal cavity, or esophagus and gastrointestinal tract.

The Lactobacillus plantarum APsulloc 331261 was deposited at Korean Culture Center of Microorganisms on Mar. 28, 2011 under Microorganism Accession No.: KCCM11179P.

Hereinafter, the configurations and effects of the present specification will be described in more detail with reference to examples, experimental examples, and formulation examples. However, these examples are provided only for the purpose of illustration to aid the understanding of the present specification, and the category and scope of the present specification are not limited by the following examples.

### [Example 1] Isolation of Lactobacillus plantarum APsulloc 331261

200 g of leaves of Camellia sinensis are washed twice with distilled water to remove foreign substances. Moisture is cleared off from the washed leaves of Camellia sinensis. Then, the leaves of Camellia sinensis are mixed with table salt corresponding to 8% by weight based on the weight of the tea leaves and left at room temperature for 3 hours. The salted leaves of Camellia sinensis are mixed with 1000 mL of a 1% fructo oligosaccharide solution, and then incubated in an incubator at 32° C. for 3 days. After 3 days, whether pH of the culture solution is lowered to less than 5 is checked, and in the case where the pH is less than 5, the culture solution is collected and incubated in Difco Lactobacilli MRS Agar® medium. At this time, the incubation is conducted in an anaerobic chamber at 32° C. for 2 days, and then white colonies are collected.

Through the method described above, the Lactobacillus plantarum APsulloc 331261 was isolated from the leaves of Camellia sinensis. The strain isolated as such was found to be a novel Lactobacillus plantarum strain as confirmed in the specification of Korean Patent No. 10-1719197 (Mar. 17, 2017). In addition, as can be understood from the contents of the application specification, it was found that the Lactobacillus plantarum strain is excellent in acid resistance and bile acid resistance, and thus can be used as medicines or foods, etc., and that even adults or infants, who are susceptible to lactic acid, may freely intake the strain.

The Lactobacillus plantarum APsulloc 331261 isolated as such was deposited at Korean Culture Center of Microorganisms on Mar. 28, 2011 under Microorganism Accession No.: KCCM11179P.

### [Experimental Example 1] Evaluation of the Effect of Inhibiting Alcoholic Gastric Damage

Four-week-old ICR mice were allowed to adapt and stabilize for one week, and then were divided into 5 groups with 8 mice in each group as follows. Culture solution with 1×10⁹ CFU of L. plantarum APsulloc 331261, culture solution with 1×10⁹ CFU of L. plantarum 299v, and omeprazole were administered in 13 mgk (mg per kg) for 7 days in each group. Then, after fasting for 4 hours on the last day, the mice were administered with 0.01 ml/g of 100% ethanol and sacrificed after 1 hour. The stomach, liver, blood and appendix were dissected and stored at −80° C. until analysis. One third of the dissected stomach was stored in 4% formaldehyde for tissue analysis. Each experimental group was classified as follows.

Group 1: Administration of only PBS (phosphate buffer saline) without ethanol as negative control

Group 2: Administration of ethanol and PBS as positive control

Group 3: Administration of L. plantarum APsulloc 331261 (AP) and ethanol

Group 4: Administration of L. plantarum 299V (299v) and ethanol

Group 5: Administration of omeprazole (gastric acid secretion inhibitor, OMPZ) and ethanol

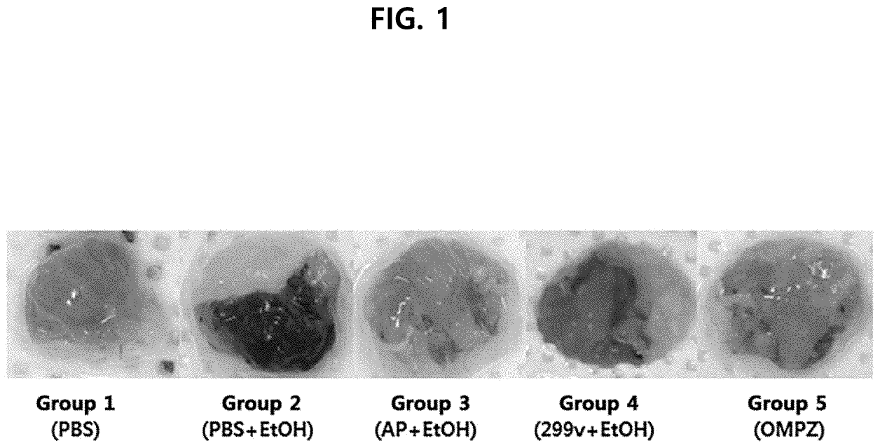

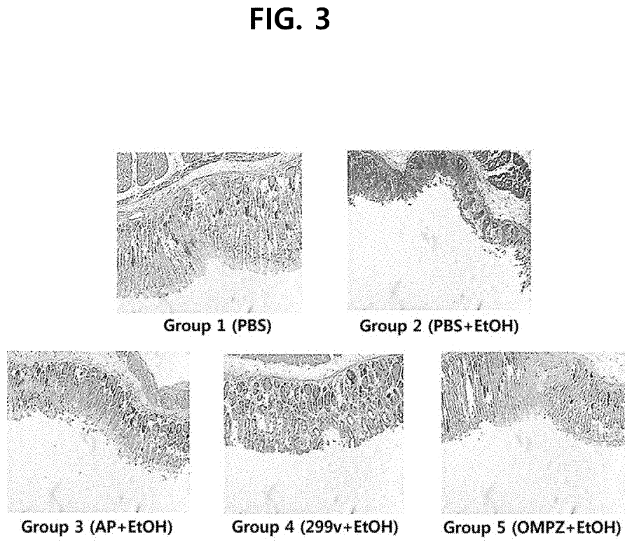

Five groups of mice were dissected, and the tissues were photographed. As a result, it was found that the ethanol-treated group showed bleeding and gastric tissue damage. However, the groups treated with ethanol and L. plantarum APsulloc 331261, L. plantarum 299V, or omeprazole along with ethanol showed less bleeding and gastric tissue damage. In particular, it was found that the administration of L. plantarum APsulloc 331261 visibly showed significantly less bleeding and gastric tissue damage than the standard strain, L. plantarum 299V, and showed similar efficacy to omeprazole, which is a gastric acid secretion inhibitor (FIGS. 1 and 3).

### [Experimental Example 2] IL6/IL10 Ratio Measurement

The expression levels of inflammatory cytokine IL6 and anti-inflammatory cytokine IL10 were confirmed in the stomach tissues. Specifically, one-third of the stomach tissue was homogenized in Trizol for 20 seconds, and then RNA was extracted according to the Ambion™ TRIzol™ Reagent Protocol. Thereafter, the purity and amount of the extracted RNA were measured using SPECTROstar Nano (BMG Labtech, Durham, USA) (2000 ng RNA, 50 ng oligo dT, 1 ul reverse transcriptase (GoScript, Promega)), and then reverse-transcribed with cDNA. Then, real time PCR was performed using takara RR820 SYBR Master Mix and ABI's stepOne plus. RNA quantitative analysis was normalized based on GAPDH and analyzed by delta-delta CT method. The primer information of the indicators used in the analysis is as follows.

TABLE 1

| Primers | Forward | Reverse |
|---|---|---|
| GAPDH | TGTGTCCGTCGTGGATCT | CCTGCTTCACCACCTTCT |
| | GA | TGA |
| IL6 | CTGCAAGAGACTTCCATC | AAGTAGGGAAGGCCGTGG |
| | CAGTT | TT |
| IL10 | TGCTATGCTGCCTGCTCT | CGGTTAGCAGTATGTTGT |
| | TAC | CCAG |

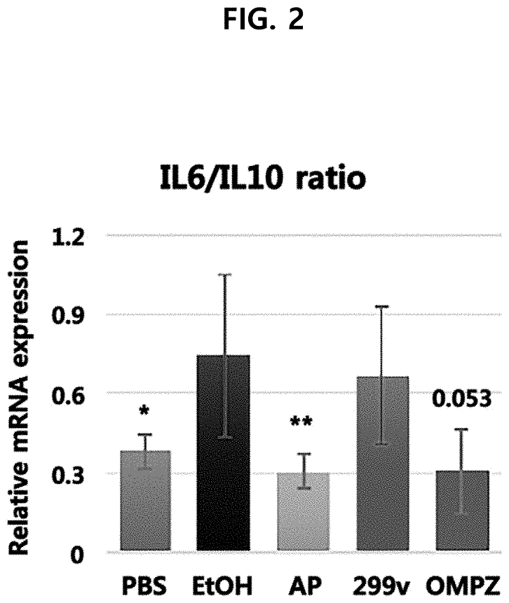

As a result, the IL6/IL10 ratio of the L. plantarum APsulloc 331261-treated group (AP) was significantly decreased in the L. plantarum APsulloc 331261-treated group compared to the EtOH control group (EtOH). This indicates that the efficacy is significantly superior to L. plantarum 299V (299v), and even better than gastric acid inhibitor of omeplazole (OMPZ). The IL6/IL10 ratio is an indicator known to increase under the gastric stress situations such as gastrointestinal ulcers including esophagitis, duodenitis, gastritis, and gastric ulcer, etc., and gastric cancer. Hence, L. plantarum APsulloc 331261 has been found to prevent, improve, or treat such disorders (FIG. 2).

Hereinafter, formulation examples of the composition are described, but they are not intended to limit one aspect of the present disclosure but merely to explain in detail.

### [Formulation Example 1] Powder

According to the preparation method of powder in the Korean Pharmacopoeia, a powder containing 20 mg of culture solution containing 10⁷ to 10⁹ cfu of L. plantarum APsulloc 331261 per pouch, 20 mg of lactose, and 10 mg of talc was prepared.

### [Formulation Example 2] Tablet

According to the preparation method of tablet in the Korean Pharmacopoeia, a tablet containing 20 mg of culture solution containing 10⁷ to 10⁹ cfu of L. plantarum APsulloc 331261 per tablet, 100 mg of corn starch, 100 mg of lactose, and 2 mg of magnesium stearate was prepared.

### [Formulation Example 3] Health Functional Beverage

According to a conventional method for preparing health functional beverage, 20 mg of culture solution containing 10⁶ to 10⁹ cfu of L. plantarum APsulloc 331261, 1000 mg of citric acid, 80 g of oligosaccharide, 2 g of plum concentrate, 1 g of taurine, milk, and purified water were added and mixed to a total volume of 900 ml. Then, the mixture was filtered, collected in a sterilized 2° C. container, sealed, and refrigerated.

Although the contents of the raw materials are a preferred example of ingredients relatively suitable for favorite beverages, the contents thereof may be appropriately modified according to hierarchy of demand priorities, consumer nations, intended use, and regional or national preferences.

[Accession No.]

Name of Depositary Authority: Korean Culture Center of Microorganisms

Accession No.: KCCM11179P

Accession Date: 20110328

1. A method for preventing, improving, or treating gastrointestinal disorders comprising administering at least one selected from the group consisting of the APsulloc 331261 (accession number: KCCM11179P) strain of Lactobacillus plantarum, a lysed product thereof, a culture thereof, and an extract thereof to a subject in need thereof.

2. The method according to claim 1, wherein the gastrointestinal disorder is an esophageal, gastrointestinal or duodenal disorder caused by alcohol.

3. The method according to claim 2, wherein the alcohol is ethanol.

4. The method according to claim 1, wherein the gastrointestinal disorder is one or more selected from the group consisting of gastritis, gastric ulcer, esophagitis, esophageal ulcer, duodenitis, and duodenal ulcer.

5. The method according to claim 1, wherein the gastrointestinal disorder is one in which the ratio of IL6/IL10 expression level in tissues of the stomach or duodenum is higher than that of a normal person.

6. The method according to claim 1, wherein a daily dosage of the APsulloc 331261 (accession number: KCCM11179P) strain of Lactobacillus plantarum is 1×10³ CFU/day to 1×10¹³ CFU/day or a daily dosage of the one or more selected from the group consisting of the lysed product, the culture, and the extract is a daily dosage obtained from the APsulloc 331261 (accession number: KCCM11179P) strain of Lactobacillus plantarum in an amount of 1×10³ CFU/day to 1×10¹³ CFU/day.

7. The method according to claim 1, wherein the one or more selected from the group consisting of the APsulloc 331261 (accession number: KCCM11179P) strain of Lactobacillus plantarum, a lysed product thereof, a culture thereof, and an extract thereof is administered in a form of a composition and the amount of the one or more selected from the group consisting of the APsulloc 331261 (accession number: KCCM11179P) strain of Lactobacillus plantarum, a lysed product thereof, a culture thereof, and an extract thereof in the composition is 0.01% by weight to 10% by weight based on the total weight of the composition.

8. The method according to claim 1, wherein the one or more selected from the group consisting of the APsulloc 331261 (accession number: KCCM11179P) strain of Lactobacillus plantarum, a lysed product thereof, a culture thereof, and an extract thereof is administered via oral administration.

9. The method according to claim 1, wherein the one or more selected from the group consisting of the APsulloc 331261 (accession number: KCCM11179P) strain of Lactobacillus plantarum, a lysed product thereof, a culture thereof, and an extract thereof is administered in a form of a composition and the composition is a food composition, a health functional food composition, or a pharmaceutical composition.

10. The method according to claim 9, wherein the formulation of a food composition, the health functional food composition, or the pharmaceutical composition is a powder or a liquid.
